(19) United States
(12) Patent Application Publication
(10) Pub. No.: US 2019/0038355 A1
(43) Pub. Date: Feb. 7, 2019

(54) END FIRE FIBER ARRANGEMENTS WITH IMPROVED EROSION RESISTANCE

(71) Applicant: Joe Denton Brown, Panama City Beach, FL (US)

(72) Inventor: Joe Denton Brown, Panama City Beach, FL (US)

(21) Appl. No.: 15/978,687

(22) Filed: May 14, 2018

Related U.S. Application Data
(60) Provisional application No. 62/506,115, filed on May 15, 2017.

(51) Int. Cl. A61B 18/24; A61B 1/018; G02B 6/36

(52) U.S. Cl. CPC A61B 18/245; A61B 2017/0034; G02B 6/3624; A61B 1/018

(57) ABSTRACT

An end-firing optical fiber arrangement includes a protective ferrule having a rounded end or a spherical fiber tip in which a notch is formed such that a planar end face of the fiber is set back from the rounded distal end of the protective ferrule or spherical fiber tip to prevent contact between the end face of the fiber and a stone or tissue.

This application claims the benefit of U.S. Provisional Patent Application Ser. No. 62/506,115, filed May 15, 2017.

### BACKGROUND OF THE INVENTION

### 1. Field of the Invention

This invention relates to optical fibers used in laser treatment applications such as, by way of example and not limitation, stone removal or lithotripsy.

The invention provides an improvement to the end-fire optical fiber arrangements with improved erosion resistance described in the inventor's U.S. patent application Ser. No. 13/692,512, filed Dec. 3, 2012, and Ser. No. 15/417,934, filed Jan. 27, 2017, each of which is incorporated herein by reference.

The improvement is that end faces of the fibers illustrated in the above-referenced copending patent applications are recessed or set back from the distal end of either the “ball tip” or the protective ferrules shown in the copending patent applications. By recessing the fiber end face, the end face is preventing from contacting a stone or other tissue, resulting in reduced erosion. The principle of providing a recess to prevent contact is also described in copending PCT Patent Appl. Ser. PCT/US2017/31091, filed May 4, 2017, but the copending PCT application achieves the recess by providing a cylindrical soft tip, rather than recess a ball tip of the type disclosed in U.S. patent application Ser. No. 13/692,512, or a glass, quartz, or sapphire ferrule of the type disclosed is U.S. patent application Ser. No. 15/417,934.

### 2. Description of Related Art

FIG. 1A shows a conventional optical fiber having a core 5 and cladding 1. End of the optical fiber is a polished flat tip 10.

FIG. 1B shows the same fiber as FIG. 1, but after laser interaction with a stone or tissue. The polished flat tip 10 of the fiber has now eroded to form an irregular surface 20 with sharp edges that could damage an endoscope's working channel.

FIG. 2A shows the arrangement disclosed in parent U.S. patent application Ser. No. 13/692,512, in which a ferrule 32 is welded to a fiber 15 to create end-firing fiber assembly 30. The end of the fiber assembly 30 is a polished flat or rounded surface 35. As described in U.S. patent application Ser. No. 13/692,512, the ferrule may be a quartz ferrule having an index of refraction that is matched to that of the fiber. In addition, the core 3 and cladding are illustrated as having an outward taper, i.e., the diameter of the fiber has been expanded in a radially outward direction towards the end surface 35, as described by Hutchens et al. in the article “Hollow Steel Tips for Reducing Distal Fiber Burn-Back During Thulium Fiber Laser Lithotripsy, Journal of Biomedical Optics 18(7), 078001 (July 2013).

Both the use of a quartz ferrule and outwardly tapered fiber illustrated in FIG. 2A have the effect of reducing erosion following interaction with a stone or tissue. Nevertheless, as illustrated in FIG. 2B, which shows laser assembly 30 following laser interaction with a stone or tissue, the eroded surface 36 is still sufficiently jagged to cause damage to the endoscope's working channel.

FIG. 3A shows a modification of the fiber shown in FIGS. 1A and 1B. In this arrangement, the fiber tip assembly 40 has a ball or spherical shape, forming a round surface with no edges to damage an endoscope, as described by Peter Kronenberg et al. in the article entitled “Lithotripsy Performance of Specially Designed Laser Fiber Tips,” which was accepted for publication in The Journal of Urology on Oct. 25, 2015. Again, the core and cladding 3 have been expanded in a radially outward direction toward the tip of the fiber, i.e., outwardly tapered, so that the outer edge 7 of the fiber tip has an expanded diameter. Nevertheless, as illustrated in FIG. 3B, erosion of the fiber tip 40 can still result in a sharp or jagged eroded surface 46 with the potential to damage the working channel of the endoscope.

### SUMMARY OF THE INVENTION

According to one aspect, the present invention further modifies the ferrule arrangement of FIGS. 2A and 2B and the ball tip arrangement of FIGS. 3A and 3B, by combining a ferrule having a rounded or spherical shape to protect the working channel of an endoscope with an optical fiber having a recessed planar end surface

### BRIEF DESCRIPTION OF THE DRAWINGS

FIGS. 1A and 1B are side views of a conventional end-firing optical fiber, respectively taken before and after laser interaction with a stone or tissue during a surgical procedure.

FIGS. 2A and 2B are side views of the ferrule-protected end-firing optical fiber assembly disclosed in U.S. patent application Ser. No. 13/692,512, respectively taken before and after laser interaction with a stone or tissue.

FIGS. 3A and 3B are side views of a conventional ball tip fiber arrangement, respectively taken before and after laser interaction with a stone or tissue.

FIGS. 4A and 4B are side views of the end-firing optical fiber arrangement with recess fiber end face of a preferred embodiment of the invention, respectively taken before and after interaction with a stone or tissue.

FIG. 4C is a side view showing a variation of the optical fiber arrangement of FIGS. 4A and 4B, in which a gap is provided between the ferrule and the tip of the fiber.

FIGS. 4D and 4E are side views of an alternate optical fiber arrangement in which a ball tip of the type shown in FIGS. 2A and 2B is provided with a recessed end face to prevent stone or tissue contact, both before and after such contact has occurred.

FIGS. 5 and 5B are side views illustrating two steps of a preferred method of assembling a protective ferrule to an optical fiber without welding.

FIG. 6 shows another alternative optical fiber arrangement, in which a protective tip is provided at the end of a sheath, so that the setback or recess can be adjusted.

FIG. 7 is a side view of ferrule of the type shown in FIGS. 4A and 4C, depicting the stripped portion of the fiber extending from a buffer and/or fiber jacket.

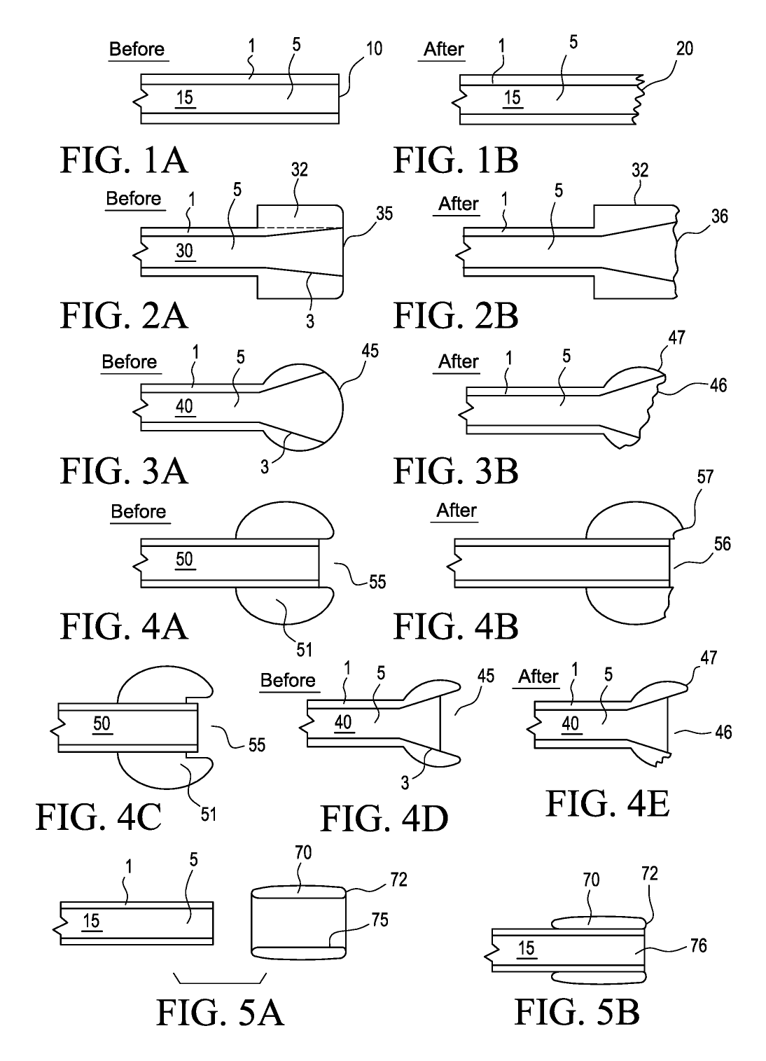

### DETAILED DESCRIPTION OF THE PREFERRED EMBODIMENTS

FIGS. 4A and 4B show a preferred embodiment of the present invention that includes an end-firing optical fiber 50 and a ferrule 51 secured to the cladding at a distal end of the fiber.

As illustrated in FIG. 4A, the ferrule 51 has a generally spherical or rounded shape, but the end surface 55 of the fiber is recessed or set back from the distal end of the fiber. The fiber core and cladding have not been tapered or expanded in a radially outward direction, in contrast to the fibers shown in FIGS. 3A and 3B. Instead, the diameter 11 of the core and cladding remains constant over the length of the fiber, including at the tip of the fiber. As in U.S. patent application Ser. No. 13/693,592, the ferrule 51 may be a quartz ferrule having an index of refraction matched to that of the fiber 10, so that laser energy disperses to increase the firing angle of the laser. Alternatively, the ferrule may be made of sapphire, glass, or another suitable material.

As illustrated in FIG. 4B, in contrast to the situations shown in FIGS. 2B and 3B, the fiber end face does not come into contact with a stone or other tissue, and therefore does not erode during a treatment procedure.

To further limit effects of contact between the ferrule and a stone or tissue, the end of the fiber may be left un-welded to the fiber, or a gap may be provided between the ferrule and the fiber tip, as shown in FIG. 4C.

FIGS. 4D and 4E show an alternative, in which the setback principle is applied to a ball tip of the type shown in FIGS. 2A an 2B, by providing a frustoconical notch in the ball tip. As is apparent from FIG. 4E, even if erosion occurs in the ball tip due to contact with a stone or tissue, the fiber end face does not begin eroding until the ball tip has substantially eroded away to expose the end face to contact, which can be prevented by providing a deep enough recess or set back.

The method of adhering the ferrule to the fiber illustrated in FIGS. 5A and 5B, which is optional, begins with the step, shown in FIG. 5A, of heating the ferrule 70 so that it expands and the inner diameter 75 of the ferrule is larger than an outer diameter of the fiber 15. The ferrule 70 is then positioned relative to the fiber 15, so that fiber tip 26 is recessed with respect to the polished front surface 72 of the ferrule 70. At this time, the ferrule 70 is permitted to cool sufficiently to shrink and grip the fiber, creating a compression fit. As a result, there is no need to weld the ferrule to the fiber. A material for the ferrule that has appropriate thermal expansion properties is sapphire.

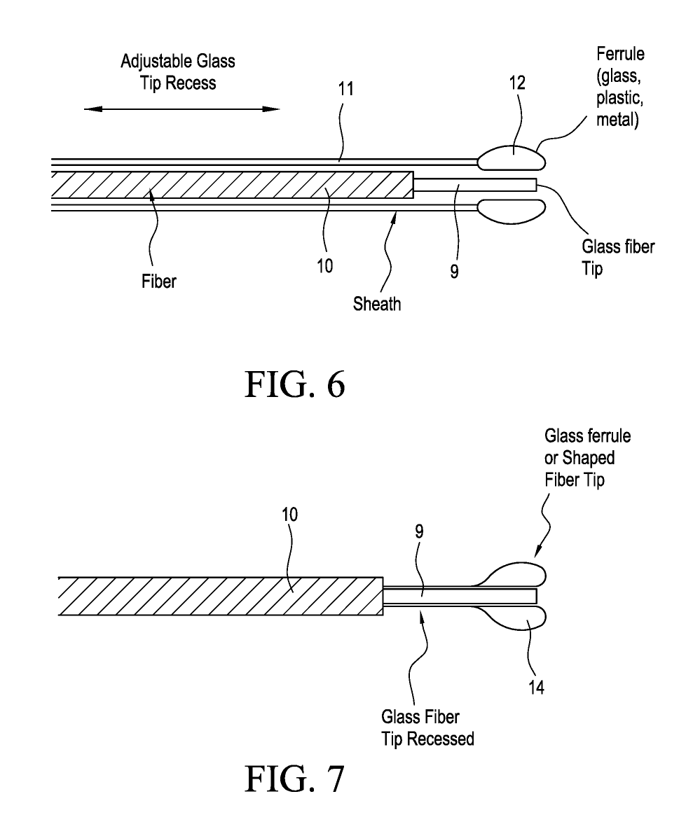

FIG. 6 shows a further variation in which the ferrule 12 is not secured to the fiber 10 at all, but rather is provided at the end of a sheath 11, so that the relative position of the fiber tip 9 and ferrule 12 can be adjusted by moving the sheath axially with respect to the fiber.

Finally, FIG. 7 illustrates the relationship between the recessed fiber tips of FIGS. 4A to 4C and a conventional stripped fiber 10 of the type shown in FIG. 6.

Having thus described preferred embodiments of the invention in connection with the accompanying drawings, it will be appreciated that the invention is not to be limited to the specific embodiments or variations disclosed.

I claim:

1. An end-firing surgical laser arrangement, comprising:
an optical fiber having an end through which laser energy exits the fiber; and
a protective ferrule having a spherical or rounded shape positioned over the end the optical fiber, or a spherical fiber tip,
wherein a planar end face of the optical fiber is set back from a distal end of the protective ferrule or spherical fiber tip,
whereby the spherical or rounded shape provides protection for the working channel of an endoscope or catheter through which the optical fiber is inserted while the contact between the planar end face of the optical fiber and a stone or tissue, thereby minimizing erosion of the end face.

2. An end-firing surgical laser arrangement as claimed in claim 1, including the protective ferrule having a spherical or rounded shape.

3. An end-firing surgical laser arrangement as claimed in claim 2, wherein the protective ferrule is made of glass, sapphire, or quartz.

4. An end-firing surgical laser arrangement as claimed in claim 2, wherein the protective ferrule is welded to the fiber, except at an end section of the fiber.

5. An end-firing surgical laser arrangement as claimed in claim 2, wherein a gap is provided between the protective ferrule and an end section of the fiber.

6. An end-firing surgical laser arrangement as claimed in claim 2, wherein the protective ferrule is provided at an end of a sheath through which the optical fiber is inserted.

7. An end-firing surgical laser arrangement as claimed in claim 1, wherein the optical fiber has a spherical tip in which a frustoconical notch is provided to form the recessed end face of the fiber.
